(19) United States
(12) Patent Application Publication
(10) Pub. No.: US 2015/0289780 A1
(43) Pub. Date: Oct. 15, 2015

(54) Method of Illumination and Illuminating Device

(71) Applicant: Thomas Benner, Erlangen (DE)

(72) Inventor: Thomas Benner, Erlangen (DE)

(21) Appl. No.: 14/683,310

(22) Filed: Apr. 10, 2015

(30) Foreign Application Priority Data
Apr. 14, 2014 (DE) 102014207108.9

(51) Int. Cl. A61B 5/055; G01R 33/30; G01B 11/27

(52) U.S. Cl. CPC A61B 5/0555; G01B 11/272; G01R 33/30

(57) ABSTRACT

Methods of illumination by laser light are provided, in particular for determining and/or adjusting the position of a patient supported on a patient table of an imaging recording device, wherein the head of the patient and/or surroundings of the head are illuminated by a laser pattern, wherein the eyes of the patient are excluded from the illumination by the laser pattern.

### CROSS-REFERENCE TO RELATED APPLICATIONS

This application claims the benefit of DE 10 2014 207 108.9, filed on Apr. 14, 2014, which is hereby incorporated by reference in its entirety.

### TECHNICAL FIELD

The embodiments relate to a method of illumination using laser light, in particular, for the purpose of determining and/or adjusting the position of a patient supported on a patient table of an imaging recording device, wherein the head of the patient and/or surroundings of the head are illuminated by a laser pattern. The embodiments further relate to an illuminating device including a laser configured to illuminate the head of a patient and/or surroundings of the head by a laser pattern.

### BACKGROUND

When preparing to examine the head of a patient by magnetic resonance tomography (MRT), a laser may be used as a guide. A laser beam is emitted by the laser and illuminates the head of the patient. The patient couch, with the patient supported thereon, may be advanced or moved until a cross-hair of the laser light illuminates a defined point on the head of the patient, e.g., between the eyes of the patient. By this, the position of the patient on the patient couch or relative to the acquisition apparatus (e.g., magnetic tube) is known, and therefore the patient may subsequently be moved to precisely the intended examination position within the tube and the examination may be performed specifically in the intended examination region of the patient.

Since the region of the marking may lie at eye level or between eye level and the forehead of the patient, (e.g., relatively close to the eyes of the patient), there is a danger that the laser light will shine directly into the eyes of the patient at a specific position of the patient table. In order to rule out any possibility of damage to the eyes of the patient due to the laser light, the patient may be prompted to close their eyes while the patient couch is set up. This is both uncomfortable and conceals the danger that the laser light will nonetheless strike the eyes, e.g., because the patient opens their eyes before the laser is switched off again.

In order to prevent this, e.g., the eyes of the patient are covered, (e.g., by the hand of a technician), during the exposure to laser light.

### SUMMARY AND DESCRIPTION

The scope of the present invention is defined solely by the appended claims and is not affected to any degree by the statements within this summary. The present embodiments may obviate one or more of the drawbacks or limitations in the related art.

The object of the embodiments is to provide a method of illumination and an illuminating device, which allow the head of a patient or the surroundings of the head to be illuminated comfortably and safely by a laser pattern, in particular, in order to align the patient supported on a patient table relative to an acquisition apparatus.

The method provides for the eyes of the patient to be excluded from the illumination by the laser pattern or laser light pattern, in particular, automatically.

In respect of the illuminating device, provision is made for the illuminating device to be configured to exclude the eyes of the patient from the illumination by the laser pattern or laser light pattern, in particular, automatically.

The eyes of the patient are specifically excluded when illuminating the patient by laser light for the purpose of positioning the patient relative to an imaging recording device, in particular, a magnetic resonance tomography system (MR device). Therefore, the eyes of the patient are not struck by the laser light at any time. It is consequently unnecessary for the patient to close their eyes or for the eyes to be covered. The illuminating device is configured accordingly to illuminate the patient away from the eyes only, and specifically to prevent any illumination of the eyes.

In a first aspect, provision is made for the laser pattern to have an inner unilluminated region for the eyes of the patient and an outer illuminated region, wherein the outer illuminated region may surround at least partially the inner unilluminated region. The laser pattern is designed to specifically exclude the region of the eyes. The laser pattern may be designed as a fixed laser pattern in this case, e.g., a laser pattern having a fixed shape. In this case, the unilluminated region may be so dimensioned that space is available for both eyes of the patient, and a predetermined safety margin may be added to this. For example, the laser pattern may have a left-hand lateral pattern region and a right-hand lateral pattern region, the separation between the lateral pattern regions being greater than a maximal eye separation in a human (e.g., between 48 and 73 mm), and a predetermined safety margin of, e.g., at least 4 centimeters is added to the maximal eye separation. Alternatively or additionally, the laser pattern may have an upper and/or a lower pattern region, wherein the pattern regions may be arranged in such a way that the eyes of the patient are not struck when the patient is positioned beneath the laser. The contiguous unilluminated region may have a height of at least 10 cm and a width of at least 20 cm, such that both eyes of a patient may be safely contained therein.

According to a second aspect, the exclusion of the eyes of the patient from the illumination by the laser pattern is effected with the aid of a head holder, in particular, a head coil of an MR device or magnetic resonance tomography system. The head holder or the head coil is arranged on the head of the patient. The laser pattern may be configured, (e.g., automatically), on the basis of the position and/or shape of the head holder, such that the laser pattern illuminates the head holder, any gaps in the head holder that are provided for the eyes of the patient being specifically excluded from the illumination.

The adaptation of the laser pattern may include changing the shape of the laser pattern. With an adaptive (e.g., dynamically varying) laser pattern (e.g., laser beam pattern), which adapts itself dynamically to the position, the type and/or the shape of the head holder or the head coil may be provided in this case. The adaptation of the laser pattern may be effected in such a way that only areas, (e.g., closed regions), of the head holder are illuminated and openings, (e.g., for the eyes of the patient), are specifically excluded from the illumination. In this way, the laser beam does not pass through the openings, but only strikes the closed regions of the holder.

Alternatively or additionally, the laser pattern may have a fixed shape and be moved integrally according to the position and/or alignment of the head holder. Provision may be made for the laser pattern to be adapted (e.g., moved) according to the so-called z-position of the patient couch (position in a z-direction, e.g., along the longitudinal patient axis). The head holder may be attached at a predetermined position of the patient couch, such that the position of the head holder relative to the patient couch is known. The movement of the laser pattern in a z-direction provides that the laser illuminates closed regions of the head holder, but no regions away from the head holder and/or no openings in the head holder. The movement of the laser pattern in a z-direction may then be entered into the calculation of the required table displacement relative to the isocenter of the recording device.

Provision may be made for a laser beam of the laser to be automatically activated, (e.g., the laser switched on), when the head holder or the head coil is in a ready mode (e.g., inserted, in standby mode) and/or in a predetermined region of the imaging recording device, in particular, in a marking region beneath the laser.

The laser pattern may be configured to exclude an eye region containing the eyes of the patient while the position of the patient is determined and/or adjusted. In this context, the eye region is predetermined by the position of the head holder or the head coil. The head holder may be mounted securely on the patient couch, or its position is otherwise known.

According to a third aspect, the laser light is generated by a laser that is switched off and/or on as a result of detecting an open and/or closed position of the eyes of the patient. If it is detected that the eyes of the patient are open, the laser is deactivated. If it is detected that the eyes of the patient are closed, the laser is activated. The detection of the open and/or closed position of the eyes may be performed by a camera that is arranged adjacent to the laser, for example. The camera, which may be located in an upper region of an opening of the imaging recording device, captures the head of the patient and, in particular, the state of their eyes. A control system may then switch the laser off and/or on automatically.

Concerning the illuminating device, in connection with the first aspect in particular, provision may be made for the laser pattern to have at least one laser line that at least partially surrounds an unilluminated region. The laser line, which may be embodied as a continuous or interrupted line, may delimit the unilluminated region at least partially relative to the exterior. For example, the delimiting laser line may be a straight or curved line that at least partially encloses the unilluminated inner region. In an embodiment variant, the inner, unilluminated region may be surrounded or enclosed at least partially by a rectangular, square, round, oval, and/or elliptic laser line in the manner of a frame.

The laser pattern may have at least one interrupted laser line, (e.g., a straight laser line), the interruption having a length that is greater than a maximal eye separation of a human. By virtue of the centrally unilluminated laser line, the eye level of the patient may be marked without illuminating the actual eyes.

Furthermore, provision may be made for the laser pattern to have at least two opposing pattern sections that are separated by an unilluminated region, the unilluminated region between the pattern sections having an extent that is greater than a maximal eye separation of a human. By virtue of the spatial separation of the illuminated regions, any illumination of and therefore damage to the eyes by the laser light may be reliably prevented.

In a further embodiment variant, the laser pattern has two laser lines, which run at an angle to each other and are interrupted in the region of their virtual intersection point. Such a centrally interrupted cross-hair allows the laser pattern to be positioned relative to the head of the patient, for example, by directing the virtual intersection point onto the region between the eyes or into a region between the eye level and the forehead of the patient. At the same time, any illumination of the eyes of the patient is reliably prevented by the centrally interrupted cross-hair. The interruption of the cross-hair may be dimensioned such that the separation between the line segments of a transverse line corresponds to at least the maximal eye separation of a human, to which may be added an additional safety margin of at least 4 centimeters, for example.

Provision is further made for a head holder or head coil that may be positioned on the head of the patient, and for an adaptation system for adapting the laser pattern such that the laser pattern illuminates the head holder, wherein any gaps in the head holder that are provided for the eyes of the patient are specifically excluded from the illumination. As explained above, the adaptation of the laser pattern may comprise an adaptation of the shape of the laser pattern and/or a movement of the laser pattern. By virtue of the dynamic adaptation of the laser pattern to the head holder, any illumination of the eyes of the patient is reliably prevented.

In connection with the third aspect, in particular, provision may be made for a detection system for detecting an open and/or closed position of the eyes of the patient, and for a control system configured to switch the laser off and/or on according to a detection signal of the open and/or closed position of the eyes. The detection system, like blink detection in modern digital cameras, detects whether the eyes of the patient are open or closed. On the basis of the corresponding detection signal (e.g., open position or closed position of the eyes), the laser is switched off and/or on via a controlled switch, for example. The laser beam is thereby reliably prevented from striking the eyes of the patient.

Finally, the embodiments relate to a recording device for recording an image data record of a patient, in particular, by magnetic resonance tomography. The recording device includes a patient table or patient couch for supporting the patient, an acquisition apparatus for acquisition of the image data record, (e.g., a region of interest in the patient), and an illuminating device for illuminating the patient, in particular, for determining and/or adjusting the position of the patient supported on the patient table. The acquisition apparatus may include a magnet with an opening into which the patient or a region of the patient may be introduced, or is designed as an open system including, for example, a C-arm or a tunnel with a lateral opening. The illuminating device may be arranged above the patient table, in order to illuminate the patient from above, particularly in the region of the head of the patient.

### BRIEF DESCRIPTION OF THE DRAWINGS

FIG. 1 depicts a first embodiment variant of a laser pattern.

FIG. 2 depicts a second embodiment variant of a laser pattern.

FIG. 3 depicts a third embodiment variant of a laser pattern.

FIG. 4 depicts a fourth embodiment variant of a laser pattern.

FIG. 5 depicts a laser pattern according to the prior art.

FIG. 6 depicts an embodiment of a recording device for recording an image data record.

Identical or corresponding components are identified by the same reference characters in all of the figures. It is also possible to combine the features that are described with reference to the individual embodiment variants and figures.

### DETAILED DESCRIPTION

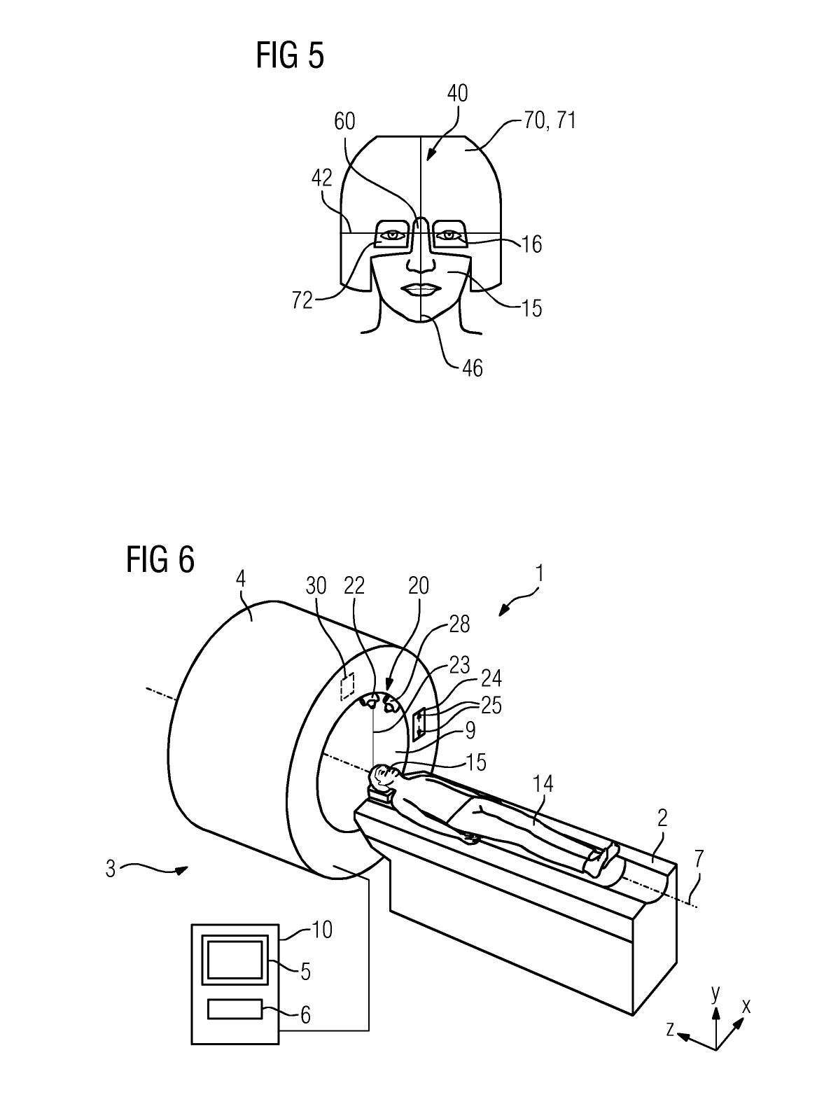

The method, the illuminating device, and the recording device are first described with reference to FIG. 6. FIG. 6 depicts a recording device 1, namely an MR device, for recording an image data record of a patient 14. The MR device features an acquisition apparatus 3 comprising a magnet 4, which may also be referred to as a magnetic tube and surrounds an examination region 9. The patient 14 lies on a patient table 2, which may be advanced along a device axis 7 (e.g., z-axis). The patient 14 may be moved into the examination region 9 by advancing the patient table 2. This is effected by corresponding motors. Additional adjustment in the x-direction and y-direction may also be provided.

The individual components of the recording device 1, (in particular, the magnet 4 and the patient table 2), are controlled by a control system 6, which may be part of an operator console 10. The control system 6 may be a computer or part of a computer, (e.g., the central arithmetic unit, such as a CPU). Provision is also made for a display screen 5, which may be part of the operator console 10.

The tubular magnet 4, which may also be referred to as the main magnet, may comprise one or more transmit and/or receive coils. A further coil, which is configured to a specific section of the examination object, (e.g., a so-called head coil 70), may also be provided in addition to this. The head coil 70, which may also be a head holder 71, may be attached to one or more predetermined positions of the patient table 2 and is intended to accommodate at least part of the head 15 of the patient 14. The head coil 70, (e.g., a receiving coil), may not enclose the head 15 of the patient 14 completely, and has openings or gaps 72 for the eyes 16, through which the patient 14 may look.

An illuminating device 20 with a laser 22 is arranged on the magnet 4, e.g., in the region of the entrance to the examination region 9. The laser 22 is configured to illuminate the patient 14 from above, in order to align the patient 14 relative to the magnet 4. For this purpose, a laser beam 23 is shone onto the head 15 of the patient 14, wherein a defined point of the head 15 is marked. For this purpose, the patient table 2, with the patient 14, is advanced along the device axis 7, wherein this may be effected by an operating unit 24 with operating buttons 25. On the basis of the known position of the laser 22, the position of the patient 14 is therefore known and the MR scan may be initialized. Instead of or in addition to moving the patient table 2, it is also possible to realize the focusing on the specific point of the patient 14 by swiveling the laser 22 in a manner that is captured by sensors.

A detection system 28 (e.g., a camera) whose function is explained in greater detail below is also provided on the magnet 4, e.g., in the region of the laser 22. An adaptation system 30 for adapting or changing the laser beam 23 may also be provided, its function likewise being described in greater detail below.

Before recording an image data record of the head 15 of the patient 14 (e.g., head MRT), the patient 14 is aligned relative to the acquisition apparatus 3, in particular, the magnet 4. For this purpose, a laser beam 23 is shone onto the head 15 of the patient 14 by the laser 22, and depicts a laser pattern 40 there. The patient 14 is then advanced by the patient table 2 until a definitive z-position is reached. For this purpose, the head 15 of the patient 14 is arranged in a predetermined position relative to the laser pattern 40. For example, the eye level of the patient 40 may be indicated by the laser pattern 40 in this case.

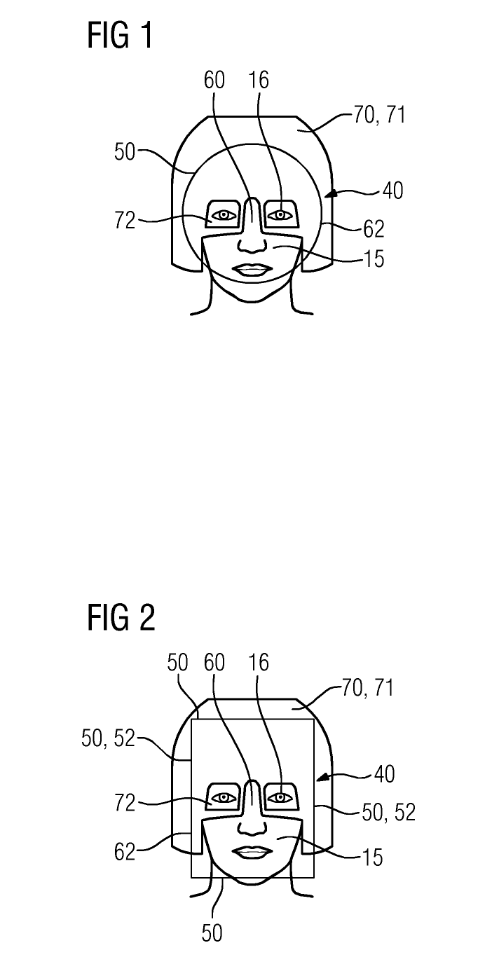

Possible laser patterns 40 of the laser 22 are described in the following with reference to the FIGS. 1 to 5. FIG. 5 depicts a laser pattern 40 according to the prior art in the form of a cross-hair. The cross-hair comprises a first laser line 42, which runs transversely relative to the patient axis or device axis 7, and a second laser line 46, which crosses the first at an angle of 90 degrees and runs along the patient axis or device axis 7. In order to align the patient 14 relative to the magnet 4, a predetermined point of the patient 14 is indicated, e.g., the region between the eyes 16 or the root of the nose. This point clearly indicates the position of the patient 14, (in particular, in a z-direction), such that the patient may subsequently be positioned precisely in the region of the isocenter of the magnet 4 in order to make a recording of the head 15.

FIGS. 1 to 4 depict exemplary embodiment variants of a laser pattern 40. The laser pattern 40 depicted in FIG. 1 includes a laser line 50 in the form of a frame that delimits the eyes 16 of the patient 14 and, for example, at least a major part of the head 15 of the patient 14. The frame is round, (in particular, circular), but may also assume a different shape such as, e.g., an oval shape. The diameter of the circle is greater than a maximal eye separation of a human, e.g., greater than 80 millimeters. In order to additionally guarantee a certain safety margin of the laser light relative to the eyes 16 of the patient 14, the diameter of the circular frame of the laser light may be at least 100 millimeters, at least 150 millimeters, or more than 200 millimeters. The laser line 50 that forms the frame is continuous. However, it is possible also to use an interrupted line, e.g., a laser line 50 including individual dots or line segments. Crucially, the laser pattern 40 has an inner region that is completely unilluminated and extends over at least both eyes 16 of the patient 14. The light beam is so dimensioned as to include a separation from the eyes 16 of the patient 14 while the patient 14 is being positioned beneath the laser 22. It is thereby provided that, even if the patient 14 is moved in a z-direction, the eyes 16 of the patient 14 are not struck by the laser beam. In order to align the patient 14 relative to the magnet 4, e.g., the eyes 16 of the patient 14 are arranged centrally relative to the z-direction in the frame of the laser pattern 40, or in another defined position relative to the laser pattern 40.

FIG. 2 depicts a further layout of the laser pattern 40 of the illuminating device 20. In a similar manner to the embodiment variant as per FIG. 1, the laser pattern 40 is embodied in the form of a frame that surrounds the eyes 16 of the patient 14. The rectangular frame is formed by a plurality of laser lines 50 and has a width that is greater than a maximal eye separation of a human, e.g., greater than the width of a human head 15. The length of the frame, e.g., its extent in a z-direction, may correspond to at least 5 or at least 10 centimeters, and may also be at least, e.g., half or all of the total length of a human head 15 or more. Furthermore, the transverse lines of the laser pattern 40 does not strike the eyes 16 if the patient table 2 is displaced by a few centimeters for the purpose of aligning the patient under the laser 22. In other respects, the explanations relating to FIG. 1 apply correspondingly.

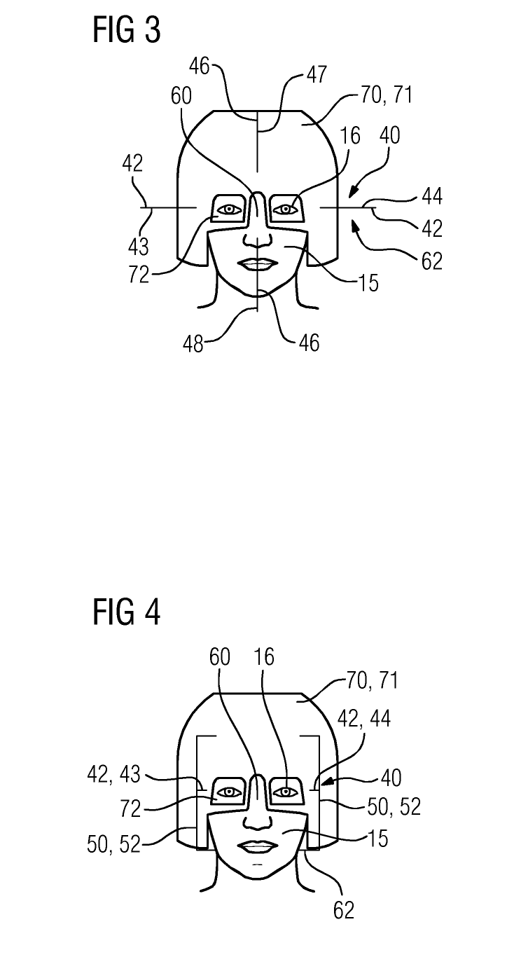

FIG. 3 depicts an embodiment variant of an laser pattern 40 that has a centrally interrupted cross-hair. The laser pattern 40 has a first interrupted laser line 42, which runs transversely relative to the device axis 7 and comprises a first line segment 43 and a second line segment 44. A second interrupted laser line 46 runs transversely to the first laser line 42, in particular parallel to the device axis 7. The second laser line 46 comprises a third line segment 47 and a fourth line segment 48. The laser lines 42, 46 intersect each other at a virtual intersection point that is located in the respective interruptions of the laser lines 42, 46 and is therefore unilluminated. This virtual intersection point may nonetheless be identified by an imaginary interpolation between the respective line segments 43, 44 and 47, 48 and placed at the predetermined reference point of the patient 14 (e.g., between the eyes, at the root of the nose). It is consequently possible to position the patient 14 precisely, while nonetheless reliably preventing any damage to the eyes 16 as a result of the laser light. The separation of the line segments 43, 44 relative to each other is greater than a maximal eye separation of a human, e.g., greater than 80 millimeters, greater than 100 millimeters, greater than 150 millimeters, or greater than 200 millimeters. The separation of the line segments 47, 48 may be greater than 100 millimeters or greater than 150 millimeters, in order reliably to prevent any illumination of the eyes 16 even in the case of a slightly oblique positioning of the head 15 of the patient 14 as a result of movement in a z-direction. It is also conceivable in principle for the second laser line 46 to be continuous, particularly if a straight alignment of the head 15 upward may be guaranteed by virtue of a head coil 70, for example.

Finally, FIG. 4 depicts an exemplary embodiment variant of a laser pattern 40 that, in particular, combines elements of the laser pattern 40 according to FIG. 2 and the laser pattern 40 according to FIG. 3. The laser pattern 40 includes lateral laser lines 52, 54 that run along the device axis 7, with small markings for marking, e.g., those regions of the head that are to be arranged in the isocenter of the recording device. Alternatively or in addition to the markings for the isocenter, provision may be made for a laser pattern that is a type of frame and delimits a region for the intended recording in a z-direction. The illustrated laser pattern 40 includes a frame that is interrupted in at least its upper and lower regions and includes lateral laser lines 52, 54 that run along the left-hand and right-hand sides of the head 15 in a longitudinal direction of the patient 14. Furthermore, the laser pattern 40 includes an interrupted laser line 42 that runs transversely relative to the device axis 7 and has line segments 43, 44. The lateral laser lines 52, 54 allow the head 15 to be positioned centrally on the device axis 7. The interrupted laser line 42 allows the patient 14 to be positioned along the device axis 7 (e.g., in a defined z-position), such that a specific z-position of the head 15, e.g., the eye level or the root of the nose, is indicated. The indication is effected by interpolation between the line segments 43, 44. The patient 14 may therefore be reliably arranged at a specific position on the z-axis by virtue of the line segments 43, 44 or markings.

The laser pattern 40 has an unilluminated region 60 that is situated in an inner region of the laser pattern 40 and extends over at least both eyes 16 of the patient 14 and the intermediate region. Outside of this unilluminated region 60, provision is made for an illuminated region 62 that is so designed that a defined relative position of the head 15 may easily be adjusted relative to the laser pattern 40 by an operator, specifically by a visual comparison of the head 15 with the laser pattern 40. For this purpose, the laser pattern 40 includes at least one marking, (e.g., a laser line), which runs transversely relative to the device axis 7 and may be positioned laterally beyond an eye 16 of the patient 14, such that the eye level or the root of the nose of the patient 14 may be indicated by extrapolation of the marking. Provision may be made for two opposing marking points or laser lines, such that the patient 14 may be positioned in a predetermined z-axis position by interpolation between these marking points.

In a further embodiment variant, in order to prevent illumination of the eyes 16, the laser pattern 40 is adapted on the basis of the position and/or shape of a head coil 70 or a head holder 71, and/or the position of the patient table 2. In order to achieve this, the laser 22 detects, e.g., the head coil 70 that has been inserted and selectively illuminates only closed regions of the head coil 70, such that the eyes 16 of the patient 14 are not struck by the laser light. The laser pattern 40 is therefore changed dynamically during the illumination of the patient 14. In this context, those regions of the laser pattern 40 that would not strike the head coil 70 are selectively switched off or excluded. For example, the laser pattern 40 may assume the basic shape of a cross-hair in this context, the cross-hair then being interrupted in the region of the openings of the head coil 70. In this way, the position of the patient 14 may be determined or adjusted particularly precisely and the laser light is reliably prevented from striking the eyes 16. In order to adapt the laser pattern 40 to the shape, position and/or alignment of the head coil 70, an adaptation system 30 may be provided, e.g., a motor for repositioning the laser 22 or the laser pattern 40.

According to a further embodiment variant, the laser is switched off if the eyes 16 of the patient 14 are open. In order to achieve this, a detection system 28 such as, e.g., a camera is provided for the purpose of detecting the open and/or closed position of the eyes 16. Such detection systems 28 are already known, e.g., so-called blink-detection facilities in the field of digital photography. The detection system 28 is activated when the head coil 70 is inserted, for example. The laser 22 is then switched off or on via the control system 6, which includes a controlled switch in particular.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method of illumination by laser light for determining, adjusting, or determining and adjusting the position of a patient supported on a patient table of an imaging recording device, the method comprising:
illuminating, by a laser pattern, a head of the patient, surroundings of the head, or the head of the patient and the surroundings of the head,
wherein eyes of the patient are excluded from the illuminating by the laser pattern.

2. The method as claimed in claim 1, wherein the laser pattern comprises (1) an inner, unilluminated region for the eyes of the patient and (2) an outer, illuminated region.

3. The method as claimed in claim 2, wherein the laser pattern is configured on the basis of a position, a shape, or the position and the shape of a head holder, such that the laser pattern illuminates the head holder, wherein any gaps in the head holder provided for the eyes of the patient are excluded from the illumination.

4. The method as claimed in claim 3, wherein the laser light is generated by a laser that is switched off or on as a result of detecting an open or closed position of the eyes of the patient.

5. The method as claimed in claim 2, wherein the laser light is generated by a laser that is switched off or on as a result of detecting an open or closed position of the eyes of the patient.

6. The method as claimed in claim 1, wherein the laser pattern is configured on the basis of a position, a shape, or the position and the shape of a head holder, such that the laser pattern illuminates the head holder, wherein any gaps in the head holder provided for the eyes of the patient are excluded from the illumination.

7. The method as claimed in claim 6, wherein the head holder is a head coil of a magnetic resonance tomography system.

8. The method as claimed in claim 6, wherein the laser light is generated by a laser that is switched off or on as a result of detecting an open or closed position of the eyes of the patient.

9. The method as claimed in claim 1, wherein the laser light is generated by a laser that is switched off or on as a result of detecting an open or closed position of the eyes of the patient.

10. An illuminating device comprising:
a laser configured to illuminate a head of a patient, surroundings of the head, or the head of the patient and the surroundings of the head by a laser pattern,
wherein the illuminating device is configured to exclude eyes of the patient from illumination by the laser pattern.

11. The illuminating device as claimed in claim 10, wherein the laser pattern comprises at least one laser line that at least partially surrounds an unilluminated region.

12. The illuminating device as claimed in claim 11, wherein the laser pattern comprises two laser lines that run at an angle to each other and are interrupted in a region of a virtual intersection point of the two laser lines.

13. The illuminating device as claimed in claim 12, wherein provision is made for a head holder configured to be positioned on the head of the patient, and an adaptation system for adapting the laser pattern such that the laser pattern illuminates the head holder, wherein any gaps in the head holder provided for the eyes of the patient are excluded from the illumination.

14. The illuminating device as claimed in claim 13, wherein provision is made for a detection system for detecting an open or closed position of the eyes of the patient, and a control system configured to switch the laser off or on as a function of a detection signal of the open or closed position of the eyes.

15. The illuminating device as claimed in claim 11, wherein provision is made for a head holder configured to be positioned on the head of the patient, and an adaptation system for adapting the laser pattern such that the laser pattern illuminates the head holder, wherein any gaps in the head holder provided for the eyes of the patient are excluded from the illumination.

16. The illuminating device as claimed in claim 10, wherein provision is made for a head holder configured to be positioned on the head of the patient, and an adaptation system for adapting the laser pattern such that the laser pattern illuminates the head holder, wherein any gaps in the head holder provided for the eyes of the patient are excluded from the illumination.

17. The illuminating device as claimed in claim 10, wherein the laser pattern comprises two laser lines that run at an angle to each other and are interrupted in a region of a virtual intersection point of the two laser lines.

18. The illuminating device as claimed in claim 10, wherein provision is made for a detection system for detecting an open or closed position of the eyes of the patient, and a control system configured to switch the laser off or on as a function of a detection signal of the open or closed position of the eyes.

19. A recording device for recording an image data record of a patient, the recording device comprising:
a patient table for supporting the patient;
an acquisition apparatus for acquiring the image data record; and
an illuminating device for illuminating the patient, the illuminating device comprising a laser configured to illuminate a head of a patient, surroundings of the head, or the head of the patient and the surroundings of the head by a laser pattern, wherein the illuminating device is configured to exclude eyes of the patient from illumination by the laser pattern.
